(19) United States
(12) Patent Application Publication
(10) Pub. No.: US 2011/0189038 A1
(43) Pub. Date: Aug. 4, 2011

(54) DRYWALL MUD PUMP WITH CLAMP OR IMPROVED FOOT VALVE

(75) Inventor: DAVID T. ROMAN, Chandler, AZ (US)

(73) Assignee: CINTA Tools Inc.

(21) Appl. No.: 12/697,825

(22) Filed: Feb. 1, 2010

(51) Int. Cl. A61M 1/00; B23P 11/00

(52) U.S. Cl. 417/437; 29/428; 222/385

(57) ABSTRACT

Drywall mud pumps for pumping drywall joint compound from buckets into drywall tools that each include a body or main cylinder, a driver or piston, and a handle. A structural component may be in rigid relation to the body and the handle may include two members that slidably or telescopically engage to allow the driver or piston to travel in a straight line while the handle pivots about a pivot point at an end of the structural component. In various embodiments, the handle may include a linear ball or PTFE bearing reducing friction between the two members, the pump may include a clamp to attach the pump to the side of the bucket, or the pump may have an inlet valve or foot valve that may include two semi-circular-shaped flaps that hingedly rotate about a pin. Methods may include acts of selecting, obtaining, or providing various such components.

### RELATED PATENT APPLICATIONS

This patent application incorporates by reference the patent application having attorney docket number 0301757-NP1, titled: DRYWALL MUD PUMP WITH IMPROVED HANDLE, having the same inventor and filed on the same date. These two patent applications have certain disclosure in common but were filed with different claims.

### FIELD OF THE INVENTION

This invention relates to pumps for pumping drywall joint compound into tools used for applying drywall joint compound between sheets of drywall.

### BACKGROUND OF THE INVENTION

Drywall, also known as gypsum board, wallboard, and plasterboard, is a building material used to finish the interior surfaces of walls and ceilings in houses and other buildings. Rigid sheets or panels of drywall are formed from gypsum plaster, the semi-hydrous form of calcium sulphate (CaSO4.½ H2O), which is typically sandwiched between two layers of heavy paper or fiberglass mats. Drywall sheets are about ½ inch thick and held in place with nails or screws to form the interior surfaces of the building, and provide fire resistance and sound deadening, among other benefits.

The joints between drywall sheets are typically filled and sealed with strips of paper or fiberglass mat and drywall joint compound, also called “joint compound”, “drywall mud”, or just “mud”. Joint compound may be made, for example, of water, limestone, expanded perlite, ethylene-vinyl acetate polymer and attapulgite. Joint compound may be applied as a viscous fluid that is thick enough to maintain its shape while it dries and hardens. In addition to forming joints, drywall mud is used to cover nail or screw heads, form a smooth or flat surface, and provide a texture over the surface. Paint or wall paper is typically applied over the drywall sheets and drywall joint compound.

Workers often specialize in the installation of drywall, and in large projects, different crews install the drywall panels (drywall hangers) from those who finish the joints and apply the joint compound (tapers or mud men). Workers who specialize in drywall installation often use specialized tools to increase their productivity. A number of tools have been invented and used for dispensing drywall joint compound. U.S. Pat. No. 7,473,085 (by Werner Schlecht), for example, describes a drywall finishing tool that is commonly referred to as a “flat box”, which is used to apply drywall joint compound between sheets of drywall, for instance. Further, drywall joint compound has been mixed at the job site in buckets, and various pumps have been used to pump the mud from the buckets into drywall tools such as flat boxes.

U.S. patent application Ser. No. 11/292,238, publication 2007/0122301 (also by Werner Schlecht) describes a drywall mud pump, for example. Various prior art drywall mud pumps used a piston in a main cylinder. In many cases, however, the piston did not travel the full length of the main cylinder. As a result, main cylinder sizes were made fairly large so that sufficient volume of drywall mud was pumped with each stroke of the pump. Further, in a number of designs, friction was excessive, making the pumps difficult to use, especially for large projects where workers have had to pump and apply a large quantity of drywall joint compound.

For these and other reasons, needs or potential for benefit exist for drywall mud pumps that are smaller in size, such as in diameter, that have less internal friction, that allow drywall mud to move freely therethrough, that have a longer piston stroke, or a combination thereof, as examples. In addition, in many prior art drywall pump designs, it was necessary to expend effort holding the pump in place while pumping drywall joint compound, which made using the pump more difficult. As a result, needs and potential for benefit exist for drywall mud pumps that do not need to be held in place while being used, as other examples. As further examples, drywall mud pumps are needed, or would be beneficial, that are inexpensive to manufacture, reliable, easy to use, that have a long life, that are easy to service and clean, and that are simple in operation so that typical operators can effectively maintain them, or that have a combination of such features. Room for improvement exists over the prior art in these and other areas that may be apparent to a person of ordinary skill in the art having studied this document. Other needs and potential for benefit may also be apparent to a person of skill in the art of specialized drywall tools.

### SUMMARY OF PARTICULAR EMBODIMENTS OF THE INVENTION

Various embodiments provide, for example, as an object or benefit, that they partially or fully address or satisfy one or more of the needs, potential areas for benefit, or opportunities for improvement described herein, or known in the art, as examples. Some embodiments of the invention provide, among other things, various apparatuses, drywall mud pumps, and methods of selecting, obtaining, providing, manufacturing, or making such devices, as examples. Drywall mud pumps, for example, may be used to pump drywall joint compound from buckets into tools for dispensing the drywall joint compound, for instance, which may then be used to apply the drywall joint compound between and/or over sheets of drywall. Workers or operators may use such drywall mud pumps, for example, who specialize in the installation of drywall, or specifically, those who finish the joints and apply the joint compound (tapers or mud men), for instance. Various embodiments provide, for example, as an object or benefit, that they provide specialized drywall mud pumps, for instance, to increase the productivity of such workers.

A number of embodiments provide, for example, as objects or benefits, adaptations and improvements to drywall mud pumps in which a piston may travel a greater length of the main cylinder. As a result, in certain embodiments, main cylinder sizes may be smaller while still providing sufficient volume of drywall mud with each stroke of the pump. Further, in some embodiments, friction may be reduced, making the pumps less difficult to use, especially for large projects where workers may have to pump and apply a large quantity of drywall joint compound. Further, in some embodiments, drywall mud may move more freely through the pump, in comparison with certain prior art alternatives for instance. In addition, in some embodiments, it may require less effort to hold the pump in place while pumping drywall joint compound, which may make using the pump less difficult. In particular embodiments, drywall mud pumps may not need to be held in place by the operator while being used, for examples. Moreover, particular embodiments provide, as an object or benefit, for instance, drywall mud pumps that are inexpensive to manufacture, reliable, easy to use, that have a long life, that are easy to service and clean, and that are simple in operation so that typical operators can effectively maintain them.

Benefits of various embodiments of the invention exist over the prior art in these and other areas that may be apparent to a person of ordinary skill in the art having studied this document. These and other aspects of various embodiments of the present invention may be realized in whole or in part in various drywall mud pumps as shown, described, or both in the figures and related description herein. Other objects and benefits may also be apparent to a person of skill in the art of specialized drywall tools, for example.

In specific embodiments, this invention provides various drywall mud pumps for pumping drywall joint compound from a bucket into a drywall tool. In a number of embodiments, such a drywall mud pump may include, for example, a main cylinder having a top end and an bottom end, a rod having a longitudinal axis, a first end, and a second end, and a piston which, when the drywall mud pump is assembled, is located within the main cylinder and is attached to the rod. In various embodiments, when the drywall mud pump is assembled, the second end of the rod is located within the main cylinder. Such embodiments may also include a pump head having an output aperture, and when the drywall mud pump is assembled, the pump head may be connected to the top end of the main cylinder and the rod may pass through the pump head, for example.

Various such embodiments may further include a structural component, and when the drywall mud pump is assembled, the structural component may be attached to the main cylinder or to the pump head, as examples, and may extend from the main cylinder or the pump head to a pivot point. Various such embodiments also include a handle which, when the drywall mud pump is assembled, is pivotably connected to the first end of the rod, and is pivotably connected to the structural component at the pivot point. Furthermore, such embodiments may also include a clamp configured to secure the drywall mud pump to a side of the bucket.

In various such embodiments the clamp may include a force-amplification mechanism, for example, and a contact surface to contact the exterior of the bucket. In certain embodiments, when the drywall mud pump is assembled and is installed within a bucket, the contact surface may face towards the main cylinder, for instance, to secure the main cylinder within the bucket, for example, by compressing the side of the bucket between the contact surface and the main cylinder. In some embodiments, the clamp may be a toggle clamp, for instance.

Further, in a number of such embodiments, the drywall mud pump may further include, for example, a foot valve, which, when the drywall mud pump is assembled, is attached to the bottom end of the main cylinder allowing drywall joint compound to flow into the main cylinder through the foot valve, but substantially preventing drywall joint compound from flowing out of the main cylinder through the bottom end. In particular embodiments, the foot valve may include, for example, a pin and two semi-circular-shaped rigid flaps that hingedly rotate about the pin. Moreover, in certain embodiments, the foot valve may further include, for instance, a tube, and when the drywall mud pump is assembled, the pin may extend through the tube and the tube may extend through a portion of each of the flaps.

In a number of such embodiments, the handle may include a first member and a second member, and when the drywall mud pump is assembled, the first member may be pivotably connected to the first end of the rod, and the second member may be pivotably connected to the structural component at the pivot point, for instance. Further, in various embodiments, when the drywall mud pump is assembled, the structural component may be rigidly attached to the main cylinder or to the pump head, and the first member may slidably or telescopically (or both) engage the second member, for instance, to allow the rod to travel in a substantially straight line along the longitudinal axis while the second member rotates about the pivot point. Still further, in some embodiments, the drywall mud pump may further include, for example, bearing mounted within the second member. In particular embodiments, the bearing may include PTFE or multiple balls, for example. Further, in various embodiments, when the drywall mud pump is assembled, part of the first member may fit inside the bearing, may extend into the second member, or both, as examples.

In other embodiments, the invention also provides various drywall mud pumps that include, for example, such a main cylinder, such a rod, and such a piston. In these embodiments, the piston may include, for example, at least one orifice through the piston to pass drywall joint compound when the piston is traveling downward in the main cylinder, and in various embodiments the piston may further include at least one flapper to block the at least one orifice to substantially prevent passage of drywall joint compound through the piston when the piston is traveling upward in the main cylinder. Such embodiments may further include a pump head, such as described above, and the pump head may include, for example, a seal around the rod. These embodiments may further include a structural component and a handle, such as described above, and may also include a foot valve, which, when the drywall mud pump is assembled, is attached to the bottom end of the main cylinder allowing drywall joint compound to flow into the main cylinder through the foot valve, but substantially preventing drywall joint compound from flowing out of the main cylinder through the bottom end, for example. In a number of embodiments, the foot valve may include, for example, a pin and two semi-circular-shaped rigid flaps that hingedly rotate about the pin.

Such embodiments may have other features previously mentioned for other embodiments as well. For example, in some embodiments, the foot valve may further include, for example, a tube, and some embodiments may further include a clamp, a handle, a bearing, or a combination thereof, as examples, such as described above. Further, in some embodiments, the structural component may be rigidly attached to the main cylinder or to the pump head, and the first member may engage the second member (e.g., as described above).

The invention also provides various methods, for example, of selecting, obtaining, or providing a drywall mud pump for pumping drywall joint compound from a bucket into a drywall tool. Such methods may include, for example, various acts, which may be performed in any order or in the order listed, as examples. In some such methods, these acts may include, for instance, selecting, obtaining, or providing a body that may include, for example, an inlet to take in drywall joint compound from the bucket, and an output aperture to deliver drywall joint compound to the drywall tool. Various such methods may further include acts of selecting, obtaining, or providing a driver to move the drywall joint compound through the body, and selecting, obtaining, or providing a structural component which, when the drywall mud pump is assembled, may be attached to the body and may extend to a pivot point. Such methods may also include, for further example, an act of selecting, obtaining, or providing a handle that may be connected in driving relation to the driver, may be pivotably connected to the structural component at the pivot point, and may rotate about the pivot point, for instance. Various such methods may also include an act of selecting, obtaining, or providing a clamp, for example, configured to secure the drywall mud pump to a side of a bucket.

In some embodiments, such methods may further include, for example, an act of selecting, obtaining, or providing a force-amplification mechanism and a contact surface to contact the exterior of the bucket. In various embodiments, when the drywall mud pump is assembled, for instance, the contact surface faces towards the body to secure the body within the bucket, for example, by compressing the side of the bucket, for instance, between the contact surface and the body. Further, in a number of embodiments, the act of selecting, obtaining, or providing the handle may include selecting, obtaining, or providing a first member and a second member, and when the drywall mud pump is assembled, the first member may be pivotably connected to the first end of the rod, the second member may be pivotably connected to the structural component at the pivot point.

Further still, in some embodiments, when the drywall mud pump is assembled, the structural component is rigidly attached to the body, and the first member slidably and telescopically engages the second member, for example, to allow the rod to travel in a substantially straight line along the longitudinal axis while the second member rotates about the pivot point. Additionally, various methods may further include, for example, an act of selecting, obtaining, or providing an inlet valve, which, when the drywall mud pump is assembled, is attached to the inlet of the body allowing drywall joint compound to flow into the inlet and into the body through the inlet valve, but substantially preventing drywall joint compound from flowing out of the body through the inlet. In particular embodiments, the inlet valve may include, for example, a pin and two semi-circular-shaped rigid flaps that hingedly rotate about the pin. Furthermore, in various embodiments the act of selecting, obtaining, or providing the handle may further include selecting, obtaining, or providing a (e.g., PTFE) bearing, as another example.

In addition, various other embodiments of the invention are also described herein.

### BRIEF DESCRIPTION OF THE DRAWINGS

FIG. 1 is a front view of a drywall mud pump illustrating an embodiment having a screw-type clamp;

FIG. 2 is a right side view of the drywall mud pump of FIG. 1;

FIG. 3 is a cross sectional front view of the drywall mud pump of FIGS. 1 and 2;

FIG. 4 is a cross sectional right side view of the drywall mud pump of FIGS. 1-3;

FIG. 5 is an isometric view of an embodiment of a drywall mud pump having a foot plate, showing the handle fully raised;

FIG. 6 is an isometric view of the drywall mud pump of FIG. 5 showing the handle fully lowered;

FIG. 7 is a cross sectional front view of another embodiment of a drywall mud pump, this embodiment having a toggle clamp and shown clamped inside a bucket;

FIG. 8 is a right side view of the drywall mud pump of FIG. 7 showing, among other things, a high filler and a goose neck attachment;

FIG. 9 is an isometric view of a structural component which is part of the drywall mud pump shown in FIGS. 7 and 8 and which supports the handle;

FIG. 10 is an isometric view of a first member of a handle which may be part of the drywall mud pump shown in FIGS. 7 and 8, for example;

FIG. 11 is a partial cross sectional view of a the handle of the drywall mud pump shown in FIGS. 7 and 8 with the grip and the pivot clamp omitted, showing, among other things, the other parts of the second member including the bearing;

FIG. 12 is an isometric view of the pivot clamp of the handle which is part of the drywall mud pump shown in FIGS. 7 and 8;

FIG. 13 is an isometric view of a toggle clamp which is part of the drywall mud pump shown in FIGS. 7 and 8;

FIG. 14 is a side view of the toggle clamp of FIG. 13, showing, among other things, the positions of the parts the clamp when the clamp is fully closed (clamped) and fully open (unclamped);

FIG. 15 is a side view of a bumper or spindle assembly that can be used with the toggle clamp of FIGS. 13 and 14;

FIG. 16 is an isometric view of the flappers and tube of an inlet valve or foot valve which may be part of the drywall mud pump shown in FIGS. 7 and 8, or other embodiments described herein, for example;

FIG. 17 is a top view of one of the flappers of the inlet valve or foot valve of FIG. 16;

FIG. 18 is a bottom view of the other flapper of the inlet valve or foot valve of FIG. 16;

FIG. 19 is a front or end view (e.g., taken from the angle of FIG. 7) of the flapper of FIG. 18 the inlet valve or foot valve of FIG. 16;

FIG. 20 is an isometric view of the base of the inlet valve or foot valve of FIG. 16;

FIG. 21 is a front view or side view of a rod and piston of a drywall mud pump, such as the embodiment shown in FIGS. 7 and 8;

FIG. 22 is an isometric view of the seal or cup of the piston shown in FIG. 21;

FIG. 23 is an isometric view of the stiffener of the piston shown in FIG. 21;

FIG. 24 is an isometric view of the flapper of the piston shown in FIG. 21;

FIG. 25 is a cross sectional side view of a high filler attachment for the drywall mud pump shown in FIGS. 7 and 8;

FIG. 26 is a cross sectional side view of a goose neck attachment for the drywall mud pump shown in FIGS. 7 and 8; and

FIG. 27 is a flow chart illustrating, among other things, an example of a method of selecting, obtaining, or providing a drywall mud pump, for example, for pumping drywall joint compound from a bucket into a drywall tool.

The drawings illustrate, among other things, various examples of embodiments of the invention, and certain examples of characteristics thereof. Different embodiments of the invention include various combinations of elements or acts shown in the drawings, described herein, known in the art, or a combination thereof, for instance.

### DETAILED DESCRIPTION OF EXAMPLES OF EMBODIMENTS

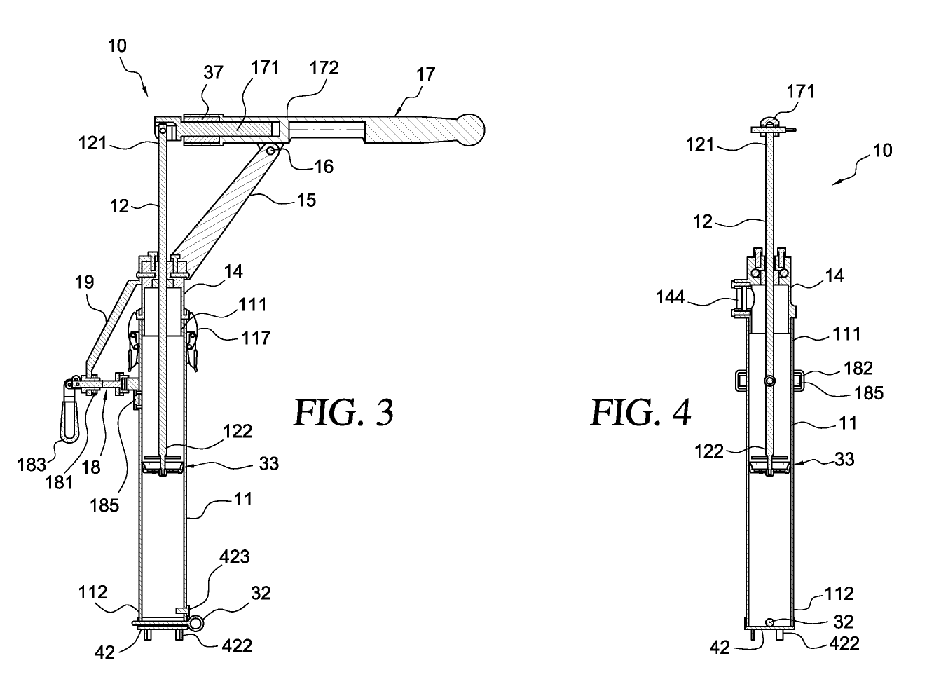

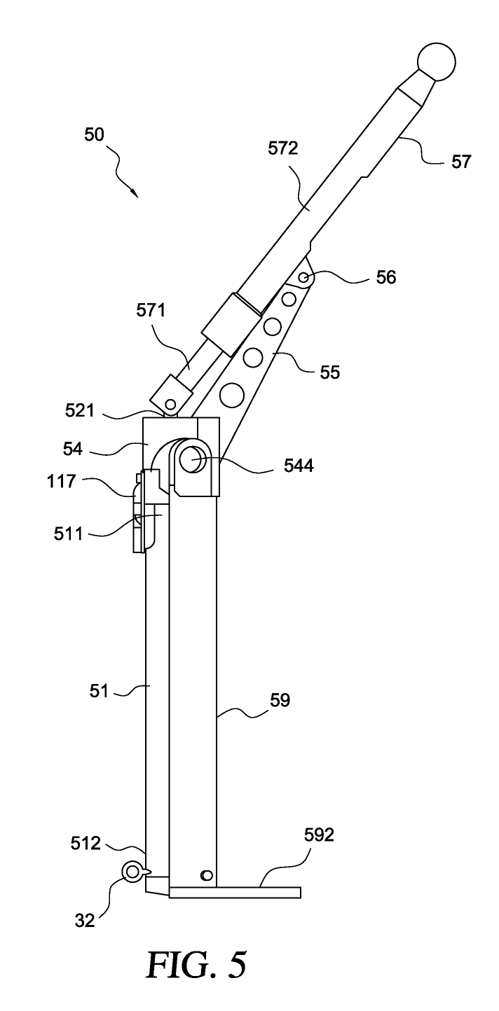

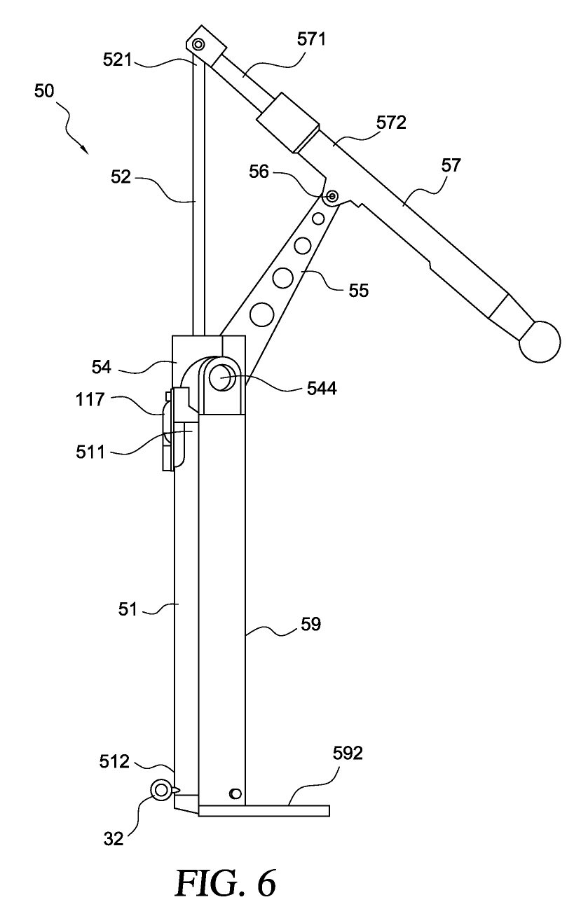

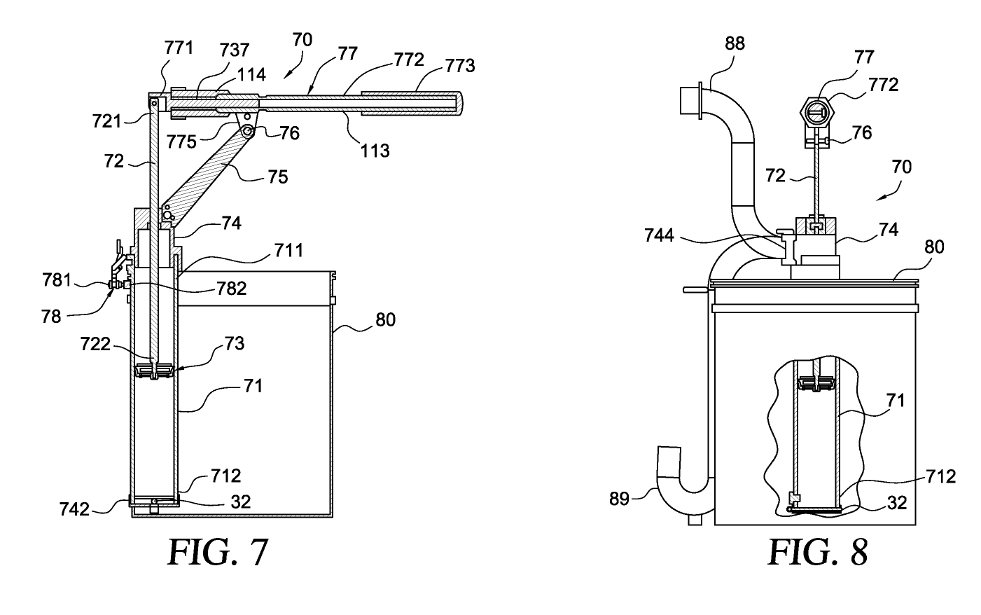

Among other things, various embodiments are, include, obtain, or provide various drywall mud pumps, for example, for pumping drywall joint compound from a bucket into a drywall tool. FIGS. 1-4 illustrate a first embodiment 10 of a drywall mud pump, FIGS. 5 and 6 illustrate a second embodiment 50 of a drywall mud pump, and FIGS. 7 and 8 illustrate a third embodiment 70 of a drywall mud pump, as examples. These figures illustrate assembled drywall mud pumps. Such pumps may be sold, shipped, or stored partially or fully disassembled, however. In a number of embodiments, a drywall mud pump may include, for example, a body, which may be or include a main cylinder (e.g., 11, 51, or 71) having (e.g., in the position shown of normal operation) a top end (e.g., 111, 511, or 711) and an bottom end (e.g., 112, 512, or 712), a rod (e.g., 12, 52, or 72) having a longitudinal axis, a first end (e.g., 121, 521, or 721), and a second end (e.g., 122 or 722), and a piston (e.g., 33 or 73 shown in FIGS. 3, 4, and 7). In a number of embodiments, when the drywall mud pump (e.g., 10, 50, or 70) is assembled, the piston (e.g., 33 or 73) may be located within the main cylinder (e.g., 11, 51, or 71) and may be attached to the rod (e.g., 12, 52, or 72). Pistons 33 and 73 are examples of drivers to move drywall joint compound through the body of drywall mud pumps 10 and 70.

In various embodiments, when the drywall mud pump (e.g., 10, 50, or 70) is assembled, the second end (e.g., 122 or 722) of the rod (e.g., 12, 52, or 72) is located within the main cylinder (e.g., 11, 51, or 71), for instance, attached to the piston (e.g., 33 or 73). Such embodiments may also include a pump head (e.g., 14, 54, or 74), for instance, which may be part of the body, having an output aperture (e.g., 144, 544, 744), and when the drywall mud pump (e.g., 10, 50, or 70) is assembled, the pump head (e.g., 14, 54, or 74) may be connected to the top end (e.g., 111, 511, or 711) of the main cylinder (e.g., 11, 51, or 71), for example, with fasteners, screws, bolts, clips, pins, or the like, as examples (e.g., clips 117 are shown for this purpose in FIGS. 1-6). In a number of embodiments, the rod (e.g., 12, 52, or 72) may pass through the pump head (e.g., 14, 54, or 74), for example. In a number of embodiments, the pump head (e.g., 14, 54, or 74) may include a seal around the rod (e.g., 12, 52, or 72), for example, an elastomeric o-ring or a u-cup, which may substantially prevent drywall joint compound from leaking out of the pump around the rod when the pump is used.

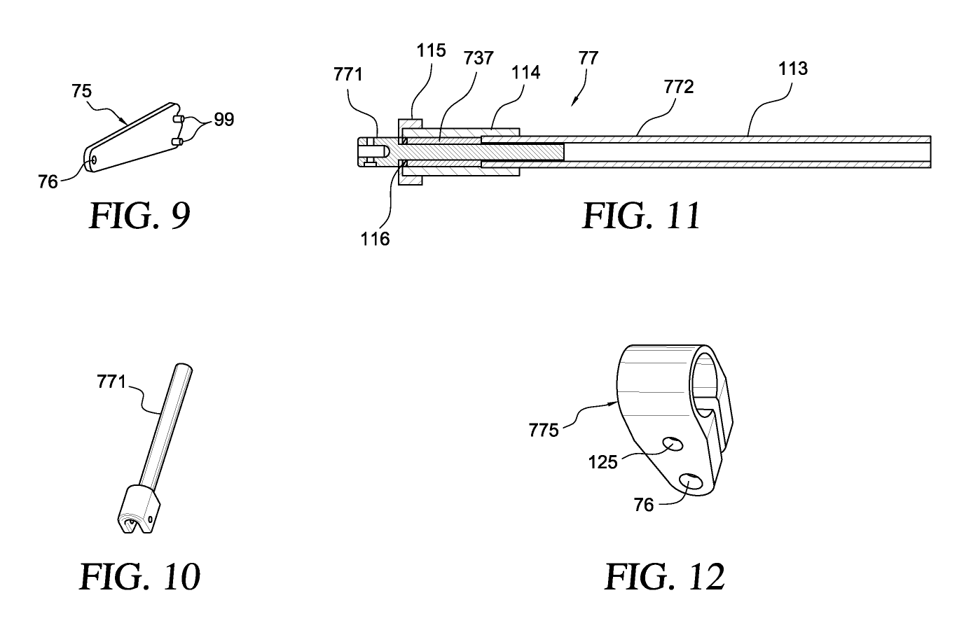

Various embodiments may further include a handle (e.g., 17, 57, or 77 shown in FIGS. 1 to 8) and a bracket or structural component (e.g., 15, 55, or 75) to support the handle of the pump. In a number of embodiments, when the drywall mud pump (e.g., 10, 50, or 70) is assembled, the structural component (e.g., 15, 55, or 75) may be rigidly attached to the main cylinder (e.g., 11, 51, or 71) or to the pump head (e.g., 14, 54, or 74), for instance, and may extend from the main cylinder (e.g., 11, 51, or 71) or the pump head (e.g., 14, 54, or 74) to a pivot point (e.g., 16, 56, or 76), for example. FIG. 9 further illustrates structural component 75 (introduced in FIG. 7) having pivot point 76 at one end and pins or fasteners 99 at the other end to attach structural component 75 to pump head 74, for example.

In various embodiments, the structural component (e.g., 15, 55, or 75) may be attached in rigid relation to the main cylinder (e.g., 11, 51, or 71) or to the pump head (e.g., 14, 54, or 74) (or both) and may extend to a pivot point (e.g., 16, 56, or 76) in rigid relation to the main cylinder (e.g., 11, 51, or 71), for example. In different embodiments, this may be accomplished by attaching the structural component (e.g., 15, 55, or 75) directly to the main cylinder (e.g., 11, 51, or 71) or to the pump head (e.g., 14, 54, or 74), or may be accomplished by attaching the structural component (e.g., 15, 55, or 75) to one or more other components that may be attached to the main cylinder (e.g., 11, 51, or 71) or to the pump head (e.g., 14, 54, or 74), or both, as examples.

As used herein, in this context, two parts being in “rigid relation” means that the parts do not move (e.g., translate or rotate) significantly relative to each other (other than due to insignificant elastic deformation of the material) while the drywall mud pump is in operation. Further, as used herein, two parts being rigidly attached to each other means that the parts are in “rigid relation”. Being “rigidly attached” or in “rigid relation” does not exclude the possibility that the two parts are detachable, for example, for disassembly, cleaning, shipping, or storage of the drywall mud pump, as examples. In fact, in many embodiments, the structural component (e.g., 15, 55, or 75) may be detachable from the pump head (e.g., 14, 54, or 74), cylinder (e.g., 11. 51, or 71), or both, for example.

In a number of embodiments, the handle (e.g., 17, 57, or 77 shown in FIGS. 1 to 8) may include, for example, a first member (e.g., 171, 571, or 771) and a second member (e.g., 172, 572, or 772). Further still, in various embodiments, when the drywall mud pump (e.g., 10, 50, or 70) is assembled, the first member (e.g., 171, 571, or 771) may be pivotably connected to the first end (e.g., 121, 521, or 721) of the rod (e.g., 12, 52, or 72), the second member (e.g., 172, 572, or 772) may be pivotably connected to the structural component (e.g., 15, 55, or 75), for instance, at the pivot point (e.g., 16, 56, or 76), or both, as examples.

In a number of embodiments, the first member (e.g., 171, 571, or 771) may slidably or telescopically engage (or both) the second member (e.g., 172, 572, or 772), for instance, to allow the rod (e.g., 12, 52, or 72) to travel in a substantially straight (e.g., vertical) line along the longitudinal axis of the rod while the second member (e.g., 172, 572, or 772) rotates about the pivot point (e.g., 16, 56, or 76). As used herein, “slidably” means that one part translates relative to another part in a substantially straight line without significant rotation therebetween. Further, as used herein, “telescopically” means that one part translates in a substantially straight line at least partially inside the other part.

Various such embodiments include a handle (e.g., 17, 57, or 77) which, when the drywall mud pump (e.g., 10, 50, or 70) is assembled, may be pivotably connected to the first end (e.g., 121, 521, or 721) of the rod (e.g., 12, 52, or 72), and may be pivotably connected to the structural component (e.g., 15, 55, or 75) at the pivot point (e.g., 16, 56, or 76). Some such handles may include the first member (e.g., 171, 571, or 771) and the second member (e.g., 172, 572, or 772) previously mentioned. FIG. 10 further illustrates first member 771 (e.g., of handle 77) introduced in FIG. 7. In the embodiments illustrated in FIGS. 1-4 and 7-8, for example, the first member (e.g., 171 or 771 of handle 17 or 77) is connected in driving relation (e.g., via rod 12 or 72) to the piston (e.g., 33 or 73), which are examples of drivers.

Further, as shown in FIGS. 3 and 7, some embodiments may include, for example, a bearing (e.g., 37 or 737), for instance, mounted within the second member (e.g., 172, 572, or 772). As used herein, where it is said that a bearing is mounted within the second member, or when the bearing is listed separately from the second member, the bearing is a separate piece from the second member and is a separate piece from the first member. In a number of embodiments, it may be possible to replace the bearing (e.g., 37 or 737)when the bearing is worn, and to reuse, for instance, the second member (e.g., 172, 572, or 772), the first member (e.g., 171 or 771), or both.

In the embodiment illustrated, when the drywall mud pump (e.g., 10 or 70) is assembled, part of the first member (e.g., 171 or 771) may fit inside the bearing (e.g., 37 or 737) and may extend into the second member (e.g., 172, 572, or 772). In a number of embodiments, the bearing (e.g., 37 or 737) may be a linear ball bearing, for example, and may include a sleeve-like outer ring and several rows of balls retained by cages. The cages or ball tracks may be oriented to provide for low friction rolling motion in a linear direction (e.g., in the axial direction). The cages or ball tracks may then curve around to return the balls to be used again. The return cages or ball tracks, and the portions of the cages and ball tracks that curve, may be deeper to prevent the balls from contacting the moving surface (e.g., first member 171, 571, or 771) when the balls are traveling in a different direction.

Other embodiments may use other types of bearings (e.g., 37 or 737) including other linear motion bearings such as linear roller bearings, roller slides, plain bearings, bushings (e.g., metal, bronze, plastic, fluoropolymer, dry lubricant, or solid lubricant), dovetail slides, or various ball slides, as examples. In the embodiments shown, the bearing (e.g., 37 or 737) is mounted inside the second member (e.g., 172, 572, or 772). In other embodiments, a bearing may be mounted on the outside of the first member. In still other embodiments, the second member may fit inside the first member, the bearing may be mounted inside the first member (or the bearing may be mounted on the outside of the second member) or a combination thereof, as examples. In some embodiments, bearings (e.g., 37 or 737) may include a low-friction material such as a fluoropolymer such as polytetrafluoroethylene (PTFE), as another example. In particular embodiments, a bearing (e.g., 37 or 737) may include PTFE filaments or fibers, which may be woven for example, and may have a greater tensile strength than PTFE resins (as another example). Various embodiments may be greaseless or self lubricating, for instance. Some embodiments may have a solid lubricant added to the wear layer. Further, some embodiments (e.g., of bearings 37 or 737) may have a fiberglass backing. Specific embodiments of PTFE bearings are available from Polygon Composites, for instance.

In certain embodiments, bearings (e.g., 37 or 737) may include Molybdenum Disulfide (MoS2), graphite, or boron nitride, as examples. In still other embodiments, bushings may be used (e.g., for bearings 37 or 737) which may be metal, such as brass, or plastic, such as nylon, which may be lubricated with a lubricant such as grease or oil, as examples. In some embodiments, openings, passageways, or fittings to apply grease or oil may be provided (e.g., on or in handle 17, 57, or 77 or in the second member 172, 572, or 772). Further, where a single bearing (e.g., 37 or 737) is shown on the drawings, other embodiments may have multiple bearings. In some embodiments, two bearings may be provided, for instance, which may be separated by a spacer or bushing. In other embodiments, a single PTFE bearing may be provided between two brass bushings, as another example. Still other embodiments may have a single longer bearing (e.g., of plastic or PTFE).

Further embodiments may lack a separate bearing. Rather, a first member (e.g., similar to 771) may slide inside and directly against a second member (e.g., similar to 772). In some embodiments, such sliding contact may be greased, for example. In such embodiments, the first member (e.g., similar to 771) and the second member (e.g., similar to 772) are not considered to be a “bearing”, as used herein. Moreover, in various embodiments, when the drywall mud pump (e.g., 10, 50, or 70) is assembled, the first member (e.g., 171, 571, or 771) may be pivotably connected to the first end (e.g., 121, 521, or 721) of the rod (e.g., 12, 52, or 72), the second member (e.g., 172, 572, or 772) may be pivotably connected to the structural component (e.g., 15, 55, or 75) at the pivot point (e.g., 16, 56, or 76), and the first member (e.g., 171, 571, or 771) may slidably engage the second member (e.g., 172, 572, or 772) through the bearing (e.g., 37 or 737), for example. Such pivotable connections may include a pin or a fastener, such as a screw or a bolt, as examples.

FIG. 11 further illustrates handle 77 that was introduced in FIG. 7. In this embodiment, second member 772 includes elongated member 113, which fits inside and is attached to bearing housing 114. In the embodiment shown in FIG. 11, bearing housing 114 contains bearing 737. Elongated member 113 may attach to bearing housing 114 with threads, welds, an adhesive, a set screw, one or more fasteners therethrough, a pin, or the like, as examples. In embodiments where elongated member 113 attaches to bearing housing 114 with threads, elongated member 113, bearing housing 114, or both, which may generally have a round cross section, may have flats formed thereon, or other features configured to receive a tool, for connecting and disconnecting elongated member 113 and bearing housing 114.

Cap 115, in this particular embodiment, attaches to bearing housing 114 and retains bearing 737 within bearing housing 114. Cap 115 may screw onto threads on bearing housing 114, for example. In other embodiments, a snap ring or interference fit may be used, as other examples. In the embodiment shown, cap 115 also retains wiper or seal 116 within bearing housing 114, for example. In this embodiment, wiper or seal 116 may be made of an elastomeric material, may be an o-ring with a round, square, or rectangular cross section, as examples, and may serve to keep dirt, drywall dust, drywall joint compound, and the like out of bearing 737, may retain grease or other lubricant within bearing 737, or a combination thereof, as examples.

As shown in FIG. 7, handle 77 also includes grip 773 which may be made of an elastomeric material and may be attached to elongated member 113, second member 772, or handle 77, for example, with an adhesive. As further illustrated in FIG. 7, and shown in detail in FIG. 12, handle 77 and second member 772 thereof also includes pivot clamp 775 which extends from elongated member 113, to pivot point 76. Pivot point 76 may include a pin or fastener (e.g., a screw or bolt) that handle 77 may rotate about when pump 70 is in use. A separate fastener may pass through hole 125 in pivot clamp 775 to tighten pivot clamp 775 around elongated member 113 to secure pivot clamp 775 in place on handle 77.

Handles 17 and 57 shown in FIGS. 1-6 may differ from handle 77 in some respects that are apparent, illustrating alternative embodiments, but may be similar in other respects. Other embodiments may differ further from these illustrated embodiments.

For instance, FIG. 11 illustrates an example or relative dimensions of the components forming handle 77. In other embodiments, the relative dimensions may differ. For example, in other embodiments, the bearing housing (e.g., 114) may be longer (e.g., about 2.5 times longer than shown), the elongated member (e.g., 113) may be shorter (e.g., about 25 percent shorter than shown), the first member (e.g., 771) may be longer (e.g., about twice as long as what is shown), or a combination thereof. Certain embodiments may have approximately these three dimensional changes specifically. Such embodiments may reduce loading on various components of handle 77, such as on the first member (e.g., 771) or the bearing(s). Other embodiments may have other dimensions changed or may change one or more of these dimensions by a different amount or in the same or different proportions. For example, in other embodiments, the bearing housing (e.g., 114) may be about 1.25, 1.5, 1.75, 2.0, 2.25, 2.75, 3.0, 3.25, or 3.5 times longer than shown (e.g., relative to other components or to the length of the handle). Further, in some embodiments, the elongated member (e.g., 113) may be about 10, 20, 30, 40, or 50 percent shorter than shown. Further still, in some embodiments, the first member (e.g., 771) may be about 1.25, 1.5, 1.75, 2.25. 2.5, 2.75, or 3.0 times as long as what is shown), as examples. In some embodiments, pivot claim 775, or a similar component, may attach to or clamp onto the bearing housing (e.g., 114) rather than to the elongated member (e.g., 113).

In various such embodiments, the bearing (e.g., 737) may be longer, (e.g., about twice as long) as what is shown. In other embodiments, the bearing (e.g., 737) may be 1.25, 1.5, 1.75, 2.25, 2.5, 3.0, or even, 3.5 longer, than what is shown (e.g., relative to other components or to the length of the handle). Further, in some such embodiments, multiple bearings may be used instead of the one bearing 737 shown. For example, in some embodiments two bearings may be separated by a busing. In a number of embodiments, a bearing may be provided at the left end of the bearing housing (e.g., 114), and a bearing (e.g., part of the same bearing or a different bearing) may be provided at a location to support the right end of the first member (e.g., 771) when the handle (e.g., 77) is at the top or bottom of its stroke, for example. In some embodiments, this location may be just to the left of the elongated member (e.g., 113), for example. On the other hand, in other embodiments, a bearing (e.g., a plastic, PTFE, or solid lubricant bearing) may be provided between two (e.g., brass) bushings (e.g., where bearing 737 is shown in FIG. 11). Such bushings may provide a replaceable wear surface. In some embodiments, such bushings (e.g., on one or both sides of a bearing) may benefit from lubrication (e.g., a solid lubricant) that carries over from the bearing.

In the illustrated embodiments, when the drywall mud pump (e.g., 10, 50, or 70) is assembled, the structural component (e.g., 15, 55, or 75) is rigidly attached to the pump head (e.g., 14, 54, or 74) and extends from the pump head (e.g., 14, 54, or 74) to the pivot point (e.g., 16, 56, or 76). The structural component (e.g., 15, 55, or 75) may be a separate piece from the pump head (e.g., 14, 54, or 74), for example, as shown, and may be attached thereto with fasteners, such as screws or bolts, for instance (e.g., pins or fasters 99 shown in FIG. 9). In other embodiments, a structural component (e.g., in lieu of 15, 55, or 75) may pivotably attach to the pump head, as another example. In the embodiments illustrated, structural members 15, 55, and 75 are formed from flat plate, for example. Other embodiments may have a different shape, such as having an I-beam cross section, being tubular (e.g., round, square, rectangular, or oval), or being an angle beam or a channel, as examples. Structural member 55 shown in FIGS. 5 and 6 illustrates that one or more cut outs or holes may be formed in structural members, for instance, to reduce the amount of material, reduce weight, provide attachment points, or the like, as examples. In some embodiments, a structural member (e.g., corresponding to 15, 55, or 75) may be a truss or may consist of two, three, four, or more sub-members, as other examples.

FIGS. 1, 3, and 7 illustrate that a number of embodiments may also include a clamp (e.g., 18 or 78), which may be configured to secure (e.g., by clamping) the drywall mud pump (e.g., 10 or 70) to a side of the bucket (e.g., 80 shown in FIGS. 7 and 8). Bucket 80 may be a common five-gallon plastic bucket, for example. In various embodiments, the clamp (e.g., 18 or 78) may include a force-amplification mechanism (e.g., 181 or 781), for example, and a contact surface (e.g., 182 or 782), for instance, to contact the exterior of the bucket (e.g., 80). In the embodiments shown, when the drywall mud pump (e.g., 10 or 70) is assembled and is installed within a bucket (e.g., 80), the contact surface (e.g., 182 or 782) may face towards the main cylinder (e.g., 11 or 71) to secure the main cylinder (e.g., 11 or 71) within the bucket (e.g., 80), for example, by compressing the side of the bucket (e.g., 80) between the contact surface (e.g., 182 or 782) and the main cylinder (e.g., 11 or 71), for instance.

FIGS. 1 and 3 illustrate that in some embodiments, the drywall mud pump (e.g., 10) further includes a clamp bracket (e.g., 19) which, when the drywall mud pump (e.g., 10) is assembled, may extend from the pump head (e.g., 14) to the clamp (e.g., 18). In embodiment 10 of the drywall mud pump, clamp bracket 19 rigidly attaches to pump head 14, and clamp 18 attaches to clamp bracket 19. In other embodiments, the clamp (e.g., 78 shown in FIG. 7) may attach directly to the pump head (e.g., 74), or the clamp (e.g., 78) may attach directly to a ring that may attach to the pump head (e.g., 74) or to the main cylinder (e.g., 71), as examples. In some embodiments, a clamp may attach directly or indirectly to the main cylinder (e.g., 11, 51, or 71). In various embodiments, the clamp may be attached such that at least part of the clamp is in rigid relation to the main cylinder, the pump head, or both, for example.

In the embodiment shown in FIGS. 1-4, clamp 18 includes an arc-shaped jaw 185 having concave surface 182 to contact the exterior of the bucket. In this embodiment, when drywall mud pump 10 is assembled, concave surface 182 faces away from force-amplification mechanism 181 and concave surface 182 faces towards main cylinder 11 for clamping main cylinder 11 within the bucket. In this embodiment, claim 18 is a screw-type clamp, and force-amplification mechanism 181 is a threaded screw mechanism that is operated by rotating handle 183. Other embodiments of force-amplification mechanisms (e.g., 781) may use leavers, cams, or the like, as other examples.

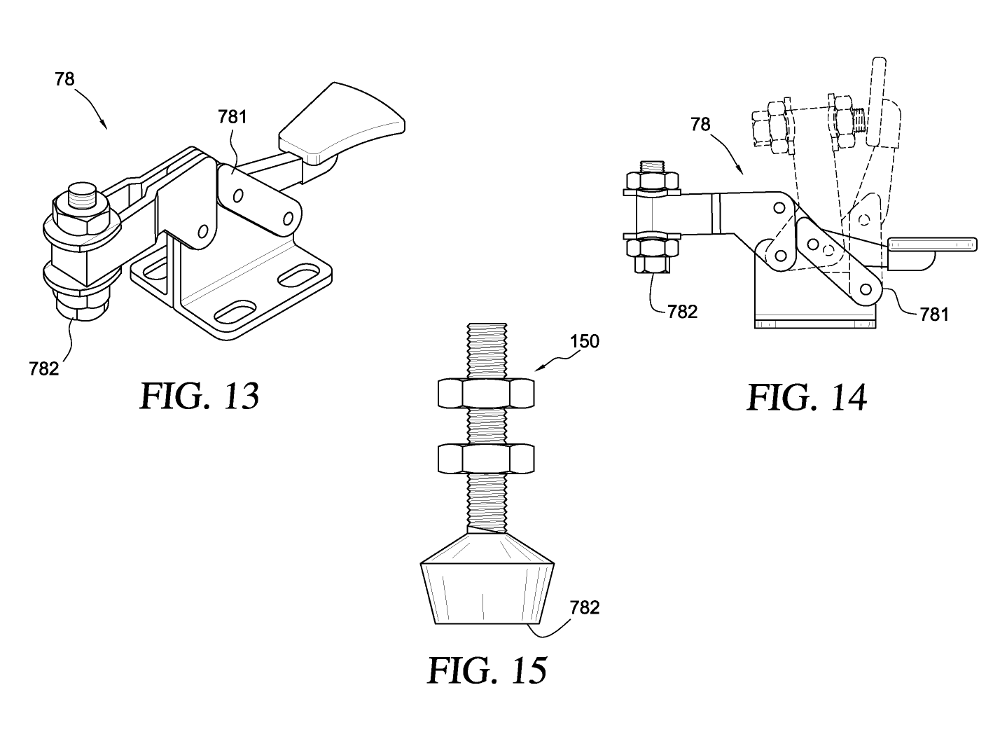

In the embodiment shown in FIG. 7, clamp 78 is a toggle clamp, for example. FIGS. 13 and 14 further illustrate toggle clamp 78, which is an example of a clamp having a force-amplification mechanism 781 that uses leavers or a linkage to provide clamping force. In different embodiments, contact surface 782 may be as illustrated in FIGS. 13 and 14 or may include an elastomeric material (e.g., a bumper) that may contact the bucket (e.g., 80), for instance. FIG. 15 illustrates an example of a bumper or spindle assembly 150 that may be used with the toggle clamp 78 shown in FIGS. 7, 13, and 14. As shown, bumper or spindle assembly 150 is adjustable so that pressure applied to the side of bucket 80 by clamp 78 can be varied, buckets (e.g., 80) with different wall thicknesses can be used, or both. Other types of clamps, toggle clamps, contact surfaces, jaws, bumpers, spindle assemblies and force-amplifications mechanisms may be used in other embodiments.

As illustrated in FIGS. 5 and 6, in some embodiments, the drywall mud pump (e.g., 50) may include a foot plate (e.g., 59) which, when the drywall mud pump (e.g., 50) is assembled, may be attached to the pump head (e.g., 54) and may extend, for example, parallel to the main cylinder (e.g., 51), for instance, at least as far (e.g., downward) from the pump head (e.g., 54) as the bottom end (e.g., 512) of the main cylinder (e.g., 51) or from the bottom of the drywall mud pump (e.g., 50). In this embodiment, rather than clamping to a bucket, the operator steps on lower portion 592 of foot plate 59 while pumping to stabilize drywall mud pump 50. Certain embodiments may have a clamp and a foot plate, as another example.

Furthermore, particular embodiments may include, for example, an inlet valve or a foot valve (e.g., 42 or 742 shown in FIGS. 1 to 4 and 7 to 8), which, when the drywall mud pump (e.g., 10, 50, or 70) is assembled, may be attached to the bottom end (e.g., 112, 512, 712) of the main cylinder (e.g., 11, 51, or 71). The bottom end (e.g., 112, 512, 712) of the main cylinder (e.g., 11, 51, or 71) or the foot valve (e.g., 42 or 742), as examples, may be, or form, an inlet to take in drywall joint compound, for example, from bucket 80. A foot valve (e.g., 42 or 742) may allow drywall joint compound to flow into the main cylinder (e.g., 11, 51, or 71), for instance, from the bucket (e.g., 80) through the foot valve (e.g., 42 or 742), but may substantially prevent drywall joint compound from flowing out of the main cylinder (e.g., 11, 51, or 71) through the bottom end (e.g., 112, 512, 712) of the main cylinder (e.g., 11, 51, or 71).

Foot valve 42 and foot valve 742 are examples of inlet valves, which, when the drywall mud pump (e.g., 10, 50, or 70) is assembled, are (one such valve is) attached to the inlet of the body (e.g., main cylinder 11, 51, or 71) allowing drywall joint compound to flow into the inlet and into the body through the inlet valve, but substantially preventing drywall joint compound from flowing out of the body through the inlet. As used herein, a foot valve (e.g., 42 or 742) “substantially” prevents drywall joint compound from flowing out of the body or main cylinder (e.g., 11, 51, or 71), for instance, through the inlet or bottom end (e.g., 112, 512, 712) if, when the drywall mud pump is in use, the amount of drywall joint compound that passes up through the inlet valve or foot valve during an upward stroke of the piston, for example, is at least twice the amount of drywall joint compound that passes downward through the foot valve during a downward stroke of the piston. Considerably better performance may be accomplished, however, in many embodiments.

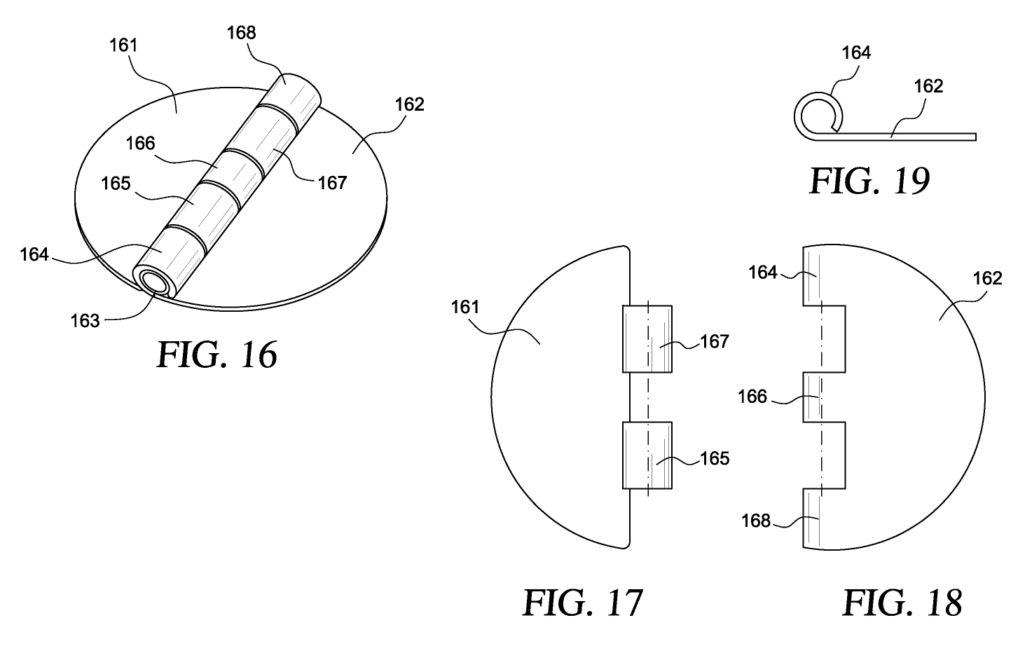

In certain embodiments, the foot valve (e.g., 42 or 742) may include, for example, a pin (e.g., 32 shown in FIGS. 1-8). In some embodiments, such a pin may have a ring at a first end, a spring detent at a second end, or both (e.g., on opposite ends), as examples. In some embodiments, two semi-circular-shaped rigid flaps may hingedly rotate about the pin (e.g., 32). FIGS. 16-19 illustrate an example of such an embodiment. In this embodiment, semi-circular-shaped rigid flaps 161 and 162 hingedly rotate about tube 163, for example, as the foot valve (e.g., 42 or 742) opens and closes. In other embodiments, corresponding flaps may have a different shape, such as rectangular. In the particular embodiment shown, the pin (e.g., 32) fits inside tube 163 and secures flaps 161 and 162 and tube 163 within the bottom end (e.g., 112, 512, or 712) of the main cylinder (e.g., 11, 51, or 71). In a number of embodiments, the pin (e.g., 32), tube (e.g., 163), or both, may extend through a portion of each of the flaps. In the specific embodiment illustrated, for example, at least when the drywall mud pump (e.g., 10, 50, or 70) is assembled, tube 163 extends through portions 164, 165, 166, 167, and 168 of flaps 161 and 162. Other embodiments may omit tube 163 or may omit the pin (e.g., 32) (or the tube may be the pin) and the tube may perform some or all of the function that the pin performs in the embodiment shown.

Flaps 161 and 162 may be metal, such as stainless steel, brass, or aluminum, or may be plastic, as examples. As used herein “rigid flaps” excludes elastomeric materials for these components. Other embodiments (i.e., not having “rigid flaps”), however, may use elastomeric flaps or a similar structure, as another example. In the embodiment shown, tube 163 may be metal, such as stainless steel, brass, aluminum, or copper, or may be plastic, such as nylon, as examples. In some embodiments, tube 163 may be made of or coated with a low friction material such as a fluoropolymer such as polytetrafluoroethylene (PTFE), as other examples.

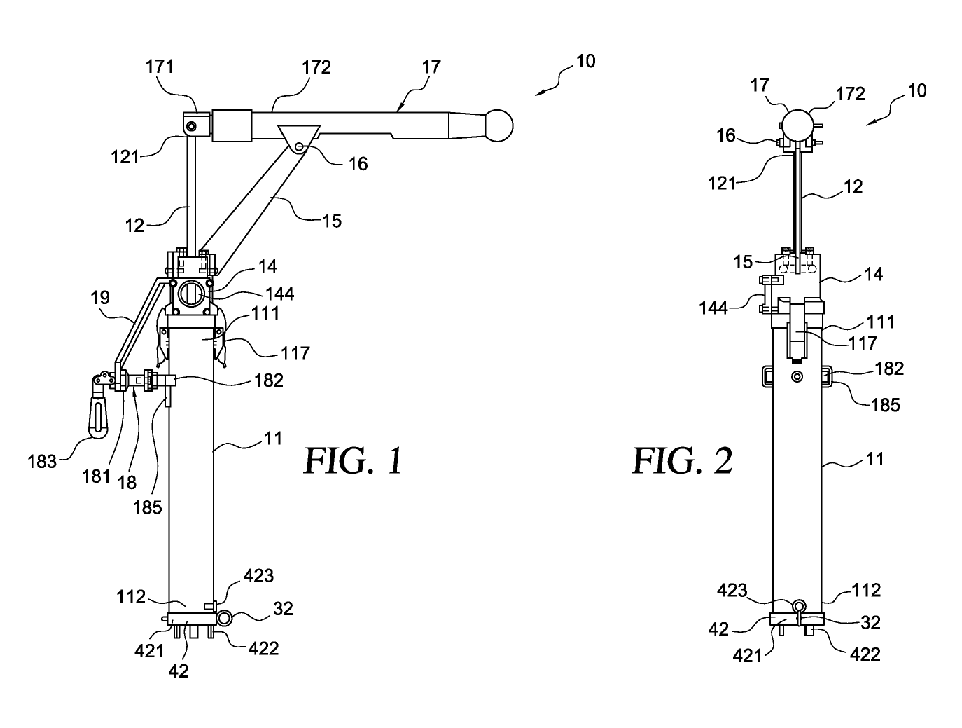

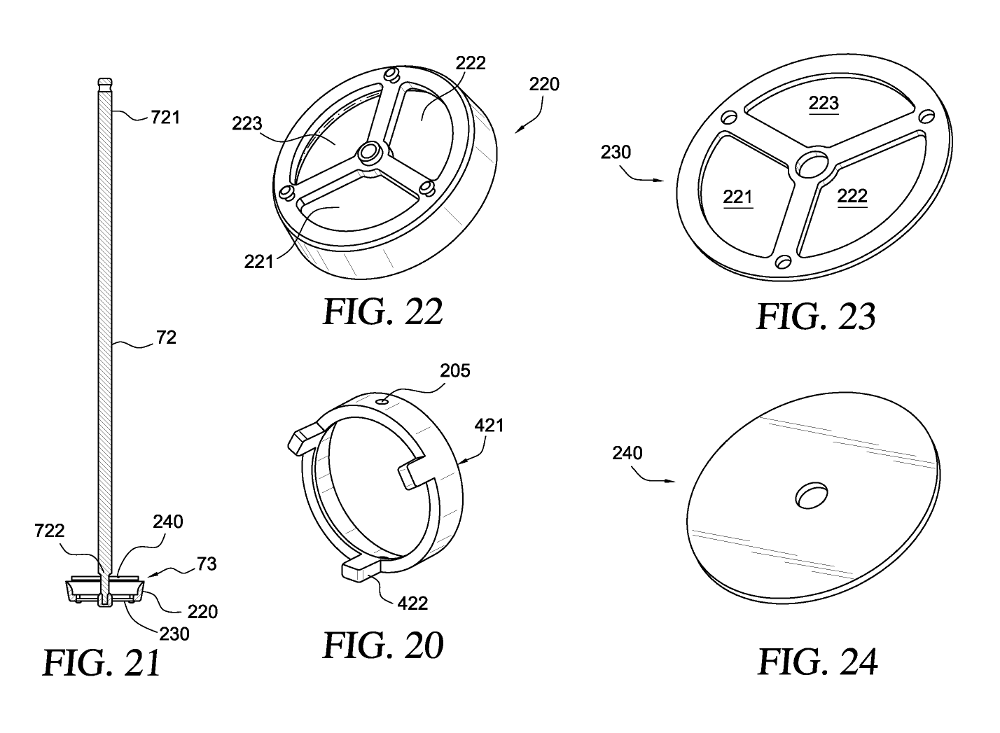

As shown in FIGS. 1 and 2, in the embodiment illustrated, pin 32 also attaches base 421 of foot valve 42 (e.g., to cylinder 11). FIG. 20 further illustrates base 421. Base 421 includes feet 422 which may provide space between bottom end 112 of main cylinder 11 and the bottom of the bucket (e.g., 80), for example, so that drywall joint compound can flow relatively freely into foot valve 42. Further feet 422 may partially or fully support drywall mud pump 10 against the bottom of the bucket. As shown in FIG. 20, base 421 includes holes 205 that pin 32 passes through to secure base 421 and foot valve 42 to bottom end 112 of main cylinder 11. Holes 205 may be large enough in diameter to pass pin 32, but too small to pass tube 163, for example.

In the embodiment illustrated, screw 423 shown in FIGS. 1-3 may act as a stop for flaps 161 and 162 preventing the flaps from raising or opening too far. This assures, in this particular embodiment, that flaps 161 and 162 close (i.e., lower) quickly and reliably when piston 33 starts its downward stroke. In many embodiments, foot valve 42 may further include a screen, for example, below flaps 161 and 162, that may serve to prevent debris and solid chunks of drywall joint compound from being drawn into drywall mud pump 10. Such a screen may be retained by or attached to base 421 of foot valve 42, in some embodiments, for example. Foot valve 742 shown in FIG. 7 may be similar to foot valve 42. In some embodiments, drywall mud pump 50 shown in FIGS. 5 and 6 may have a similar foot valve.

FIG. 21 further illustrates rod 72 and piston 73 introduced in FIG. 7. FIGS. 22-24 further illustrate various components and features of piston 73, as examples. In the embodiment illustrated, piston 73 includes, for example, elastomeric piston cup 220 shown in FIGS. 21 and 22. In various embodiments, at least one orifice exists through the piston (e.g., 33 or 73) to pass drywall joint compound when the piston (e.g., 33 or 73) is traveling downward in the main cylinder (e.g., 11, 51, or 71). As shown in FIG. 22, piston cup 220 has three orifices 221, 222, and 223 through piston cup 220. These orifices 221 to 223 line up with correspondingly numbered orifices in piston stiffener 230 shown in FIG. 23. Other embodiments may have two, four, five, six, or more orifices through the piston to pass drywall joint compound, for example, when the piston is traveling downward in the main cylinder. Piston cup 220 may be made of an elastomeric material (e.g., nitrile rubber or buna n) to provide effective sealing against cylinder 71. Piston stiffener 223, on the other hand, may be made of a stiffer material such as metal (e.g., stainless steel) or a hard plastic.

In the embodiment illustrated, orifices 221-223 extend through piston 73 to pass drywall joint compound through piston 73 when the piston is traveling downward in the main cylinder (e.g., 71). When piston 73 is traveling downward in the main cylinder, flapper 240 shown in FIGS. 21 and 24 moves away from orifices 221, 222, and 223 allowing drywall joint compound to pass through the orifices 221-223 and around the outside of flapper 240, extending the orifices 221 to 223 and providing a pathway for the drywall joint compound through piston 73. In contrast, in the embodiment illustrated, flapper 240 blocks the orifices 221, 222, and 223 to substantially prevent passage of drywall joint compound through piston 73 when the piston is traveling upward in the main cylinder (e.g., 71). As used herein, in this context, a flapper “substantially” blocks passage of drywall joint compound if more than twice as much drywall joint compound is moved upward by the piston as leaks through the piston when the piston travels upward.

Flapper 240 may be made of a stiff or moderate stiffness material, as examples. In particular embodiments, for example, flapper 240 may be made of plastic, such as nylon, for instance. In other embodiments, a piston flapper may be made of metal (e.g., stainless steel) or of an elastomeric material, as other examples. Elastomeric flappers may bend out of the way when the piston is traveling downward, facilitating flow, but may require more support (e.g., a greater number of smaller orifices) when the piston is traveling upwards.

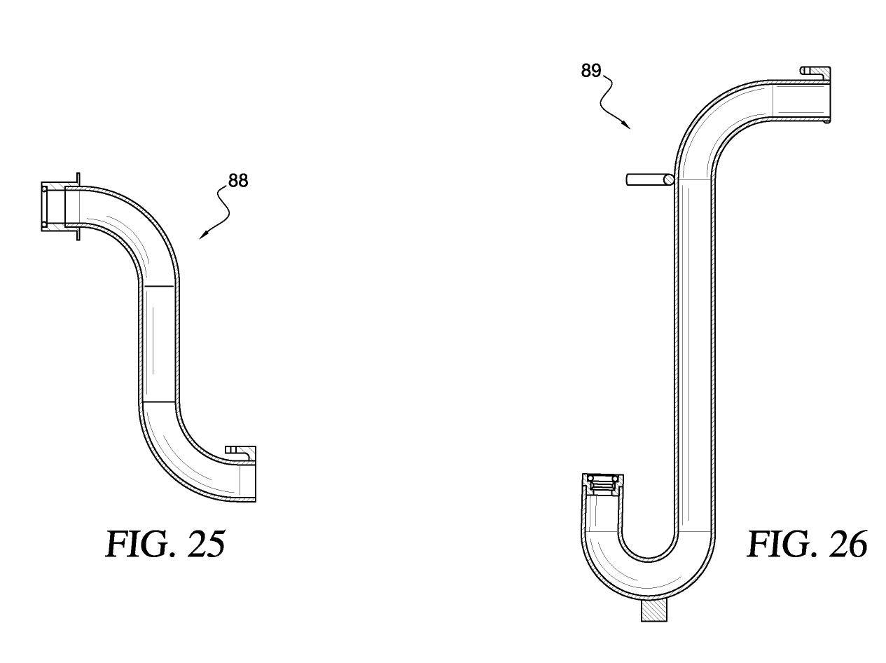

FIG. 8 illustrates that various embodiments may include a high filler (e.g., 88), a goose neck (e.g., 89), or both. Although both high filler 88 and goose neck 89 are shown in FIG. 8, only one of high filler 88 or goose neck 89 would be installed on drywall mud pump 70 at a time, in this embodiment. FIGS. 25 and 26 further illustrate high filler 88 and goose neck 89. In the embodiment illustrated, high filler 88 and goose neck 89 are each formed from a piece of tubing having a round cross section and configured at the ends of the tubing to releasably attach to pump head 74 and to a drywall tool. Where high filler 88 and goose neck 89 attach to a drywall dispensing tool, for example, high filler 88 and goose neck 89 may include a bushing, a seal such as an o-ring, or both, as examples. In the embodiment shown, high filler 88 may be used to fill a flat box, for example, without requiring the operator to bend over as far. Further, in this embodiment, goose neck 89 may be used to fill a corner tool that has a cylinder that holds drywall joint compound under pressure from a piston and spring (e.g., a pneumatic spring) for example. In some embodiments, a high filler may discharge over the bucket (e.g., 88) to avoid spills (e.g., so that spills from the high filler fall back into the bucket).

In the embodiment illustrated, high filler 88 and goose neck 89 attach (e.g., one at a time) to output aperture 744 of pump head 74. Output aperture 744, or the end of high filler 88 or goose neck 89 that attaches to the drywall dispensing tool, are examples of outlet apertures to deliver drywall joint compound to the drywall tool. Outlet apertures 144 and 544 are other such examples. In the embodiment illustrated, high filler 88 and goose neck 89 each have two bends. Specifically, high filler 88 has two 90 degree bends, and goose neck 89 has one 90 degree bend and one 180 degree bends. Other embodiments may differ.

Except where otherwise described, various components may be made of metal, for example, such as aluminum, stainless steel, brass, copper, galvanized steel, or chrome plated steel, as examples. Some components may be made of plastic, as another example, such as nylon. In other embodiments, other materials may be used, which may be selected, for example, to avoid corrosion when exposed to moist drywall joint compound. In some embodiments, some components may be coated or painted to prevent corrosion.

FIGS. 5 and 6 illustrate handle 57 in a fully raised and fully lowered position for embodiment 50 of the drywall mud pump. In various embodiments, for example, the handle may rotate between 80 and 120 degrees, or between 90 and 110 degrees, from the fully raised to the fully lowered position, as examples. In specific embodiments, for example, the handle may rotate about 100 degrees from the fully raised to the fully lowered position. As used herein, “about”, when referring to angles of handle rotation, means within plus or minus 5 degrees. In other embodiments, for further example, the handle may rotate about 50, 60, 70, 80, 85, 90, 95, 105, 110, 115, 120, 125, 130, 140, or 150 degrees from the fully raised to the fully lowered position, as other examples.

In various embodiments, the main cylinder (e.g., 11, 51, or 71 shown in FIGS. 1-8) may have an inside diameter that is between 1.5 and 4 inches, between 1.75 and 3.5 inches, between 2 and 3 inches, between 2.25 and 2.75 inches, between 2.25 and 2.5 inches, or between 2.3 and 2.5 inches, as examples. In some embodiments, the main cylinder (e.g., 11, 51, or 71 shown in FIGS. 1-8) may have an inside diameter that is about 2.4 inches, for example. As used herein, when referring to dimensions, “about” means within plus or minus two percent.

A number of embodiments include various methods, for example, of selecting, obtaining, or providing a drywall mud pump (e.g., 10, 50, or 70) for pumping drywall joint compound from a bucket (e.g., 80) into a drywall tool (e.g., a flat box). Such methods may include, for example, various acts, which may be performed in various sequences or orders, examples of which include the order listed herein or shown on the drawings.

Various methods and acts described include selecting, obtaining, or providing certain structure or components. As used herein, “selecting” includes ordering, for example, from products, components, or materials offered for sale, manufactured, or provided by others. “Selecting”, however, as used herein, does not include purely mental processes such as choosing in one's mind what structure or components to use. Further, “obtaining”, as used herein, includes purchasing such structure or components. Still further, “obtaining”, as used herein, includes manufacturing or assembling (or both) such structure or components. Even further, “obtaining”, as used herein, includes receiving such structure or components (e.g., manufactured or assembled by others). Moreover, as used herein, “providing” includes any combination of selling, advertising for sale, renting, leasing, and shipping such structure or components. Furthermore, although various embodiments are described in terms of selecting, obtaining, or providing certain structure or components, other embodiments may be limited to just selecting, just obtaining, or just providing the particular structure or components, or to just obtaining or providing such structure or components, as further examples.

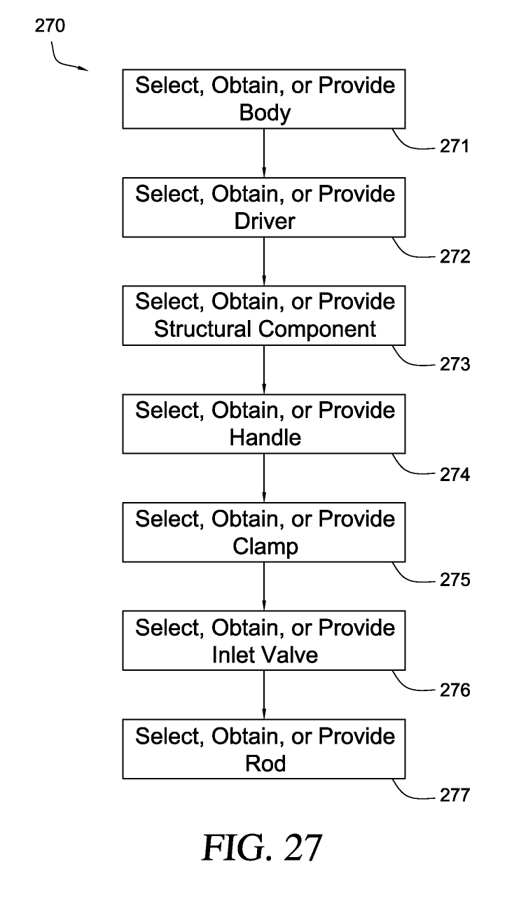

In some methods (e.g., method 270 shown in FIG. 27), an act may include, for instance, selecting, obtaining, or providing a body (e.g., act 271 shown in FIG. 27). Examples of such a body (e.g., of act 271) include main cylinder 11, 51, and 71 illustrated in FIGS. 1-8. In some embodiments, such a body (e.g., of act 271) may further include a pump head (e.g., 14, 54, or 74), for instance. In particular embodiments, such a body (e.g., of act 271) may further include other components, such as high filler 88, goose neck 89 or both (e.g., shown in FIGS. 8, 25, and 26), as examples. In a number of embodiments, such a body (e.g., of act 271) may include, for example, an inlet (e.g., bottom end 112, 512, or 712 of main cylinder 11, 51, or 71 or foot valve 42 or 72) to take in drywall joint compound from the bucket (e.g., 80), an output aperture (e.g., 144, 544, or 744 or an opening of a high filler (e.g., 88), a goose neck (e.g., 89), or both) to deliver drywall joint compound to the drywall tool, or both an inlet and an outlet.

Various such methods, including method 270 shown, may further include an act of selecting, obtaining, or providing a driver (e.g., act 272), for instance, to move the drywall joint compound through the body. Examples of such a driver (e.g., of act 272) include pistons 33 and 73 shown, for instance, in FIGS. 3, 7, and 21-24. Another act shown in method 270 is act 273 of selecting, obtaining, or providing a structural component (e.g., 15, 55, or 75 shown in FIGS. 1, 3, 5-7, and 9) which, when the drywall mud pump (e.g., 10, 50, or 70) is assembled, may be attached to the body (e.g., to main cylinder 11, 53, or 73) and may extend to a pivot point (e.g., 16, 56, or 76 shown in FIGS. 1, 3, and 5-7).

In the embodiment illustrated, method 270 also includes, for further example, act 274 of selecting, obtaining, or providing a handle (e.g., 17, 57, or 77 shown in FIGS. 1-8 and 11). Such a handle (e.g., of act 274) may specifically include, for example, a first member (e.g., 171, 571, or 771) and a second member (e.g., 172, 572, or 772). Further, in various embodiments, when the drywall mud pump (e.g., 10, 50, or 70) is assembled, the first member (e.g., 171, 571, or 771) may be connected in driving relation to the driver (e.g., piston 33 or 73, for example, via rod 12 or 72), the second member (e.g., 172, 572, or 772) may be pivotably connected to the structural component (e.g., 15, 55, or 75) at the pivot point (e.g., 16, 56, or 76), and the first member (e.g., 171, 571, or 771) may slidably or telescopically (or both) engage the second member (e.g., 172, 572, or 772) while the second member (e.g., 172, 572, or 772) rotates about the pivot point (e.g., 16, 56, or 76), for instance. In a number of embodiments, the handle (e.g., provided in act 274, for example, handle 17, 57, or 77) may be connected in driving relation to the driver (e.g., piston 33 or 73), may be pivotably connected to the structural component (e.g., 15, 55, or 75) at the pivot point (e.g., 16, 56, or 76), may rotate about the pivot point (e.g., 16, 56, or 76), or a combination thereof, for instance.

In the embodiment shown, methods 270 also includes act 275 of selecting, obtaining, or providing a clamp (e.g., 18 or 78), for example, configured to secure the drywall mud pump (e.g., 10, 50, or 70) to a side of a bucket (e.g., 80). In some embodiments, act 275 (e.g., of method 270) may further include, for example, selecting, obtaining, or providing a force-amplification mechanism (e.g., 181 or 781 shown in FIGS. 1, 7, 13, and 14) and a contact surface (e.g., 182 or 782), for example, to contact the exterior of the bucket (e.g., 80). In various embodiments, when the drywall mud pump (e.g., 10, 50, or 70) is assembled, for instance, the contact surface (e.g., 182 or 782) may face towards the body (e.g., main cylinder 11, 51, or 71), for instance, to secure the body (e.g., main cylinder 11, 51, or 71) within the bucket (e.g., 80), for example, by compressing the side of the bucket (e.g., 80), for instance, between the contact surface (e.g., 182 or 782) and the body (e.g., main cylinder 11, 51, or 71).

Further, in a number of embodiments, the act of selecting, obtaining, or providing the handle (e.g., act 274, for instance, handle 17, 57, or 77) may include selecting, obtaining, or providing a first member (e.g., 171, 571, or 771) and a second member (e.g., 172, 572, or 772). In a number of embodiments, when the drywall mud pump (e.g., 10, 50, or 70) is assembled, the first member (e.g., 171, 571, or 771) may be pivotably connected to the first end (e.g., 121, 521, or 721) of the rod (e.g., 12, 52, or 72), the second member (e.g., 172, 572, or 772) may be pivotably connected to the structural component (e.g., of act 273, for example, structural component 15, 55, or 75) at the pivot point (e.g., 16, 56, or 76).

Additionally, method 270 shown in FIG. 27 further includes, for example, act 276 of selecting, obtaining, or providing an inlet valve (e.g., foot valve 42 or 742), which, when the drywall mud pump (e.g., 10, 50, or 70) is assembled, may be attached to the inlet (e.g., bottom end 112, 512, or 712) of the body (e.g., main cylinder 11, 51, or 71) allowing drywall joint compound to flow into the inlet and into the body through the inlet valve, but substantially preventing drywall joint compound from flowing out of the body through the inlet. In particular embodiments, the inlet valve (e.g., foot valve 42 or 742) may include, for example, a pin (e.g., 32) and two semi-circular-shaped rigid flaps (e.g., 161 and 162 shown in FIGS. 16-19) that hingedly rotate about the pin.

Furthermore, in various embodiments the act of selecting, obtaining, or providing the handle (e.g., act 274, for instance, handle 17, 57, or 77) may further include selecting, obtaining, or providing a bearing (e.g., 37 or 737 shown in FIGS. 3, 7, and 11), as another example. Such a bearing may be or include PTFE, for example. Further, in various embodiments, when the drywall mud pump (e.g., 10, 50, or 70) is assembled, part of the first member (e.g., 171, 571, or 771) may fit inside the bearing (e.g., 37 or 737), may extend into the second member (e.g., 172, 572, or 772), or both, as examples. Moreover, in various embodiments the act of selecting, obtaining, or providing the driver (e.g., act 272) may specifically include selecting, obtaining, or providing a piston (e.g., 33 or 73 shown in FIGS. 3, 4, 7, and 21-24), for instance, having at least one orifice (e.g., 221, 222, 223, or a combination thereof, shown in FIGS. 22 and 23) therethrough to pass drywall joint compound through the piston (e.g., 33 or 73), having at least one flapper (e.g., 240 shown in FIGS. 21 and 24) to block the at least one orifice (e.g., 221, 222, 223, or a combination thereof) to prevent passage of drywall joint compound through the piston (e.g., 33 or 73).

Further, in some embodiments, the act of selecting, obtaining, or providing the body (e.g., act 271) may specifically include selecting, obtaining, or providing a main cylinder (e.g., 11, 51, or 71). Still further, in the embodiment shown, method 270 includes, for example, act 277 of selecting, obtaining, or providing a rod (e.g., 12, 52, or 72). In a number of embodiments, when the drywall mud pump (e.g., 10, 50, or 70) is assembled, the rod (e.g., provided in act 277) may extend from the first member (e.g., 171, 571, or 771) to the driver (e.g., piston 33 or 73), for example. Still further, in some embodiments, when the drywall mud pump (e.g., 10, 50, or 70) is assembled, the structural component (e.g., 15, 55, or 75) may be attached in rigid relation to the body (e.g., to main cylinder 11, 51, or 71), the pivot point (e.g., 16, 56, or 76) may be in rigid relation to the body, or both, as examples.

The various components shown in the different drawings, described herein, or both, may be found in various combinations in different embodiments. Other embodiments may be apparent to a person of ordinary skill in the art having studied this document, and may include features or limitations described herein, shown in the drawings, or both. Various methods may include part or all of the acts shown in FIG. 27, described herein, or known in the art, as examples.

What is claimed is:

1. A drywall mud pump for pumping drywall joint compound from a bucket into a drywall tool, the drywall mud pump comprising:
a main cylinder having a top end and an bottom end;
a rod having a longitudinal axis, a first end, and a second end, wherein, when the drywall mud pump is assembled, the second end is located within the main cylinder;
a piston which, when the drywall mud pump is assembled, is located within the main cylinder and is attached to the rod;
a pump head having an output aperture, wherein, when the drywall mud pump is assembled, the pump head is connected to the top end of the main cylinder and the rod passes through the pump head;
a structural component wherein, when the drywall mud pump is assembled, the structural component is attached to the main cylinder or to the pump head and extends from the main cylinder or the pump head to a pivot point;
a handle wherein, when the drywall mud pump is assembled, the handle is pivotably connected to the first end of the rod, the handle is pivotably connected to the structural component at the pivot point; and
a clamp configured to secure the drywall mud pump to a side of the bucket.

2. The drywall mud pump of claim 1 wherein the clamp comprises a force-amplification mechanism and a contact surface to contact an exterior of the bucket, wherein, when the drywall mud pump is assembled and is installed within a bucket, the contact surface faces towards the main cylinder to secure the main cylinder within the bucket by compressing the side of the bucket between the contact surface and the main cylinder.

3. The drywall mud pump of claim 1 wherein the clamp is a toggle clamp.

4. The drywall mud pump of claim 1 further comprising a foot valve, which, when the drywall mud pump is assembled, is attached to the bottom end of the main cylinder allowing drywall joint compound to flow into the main cylinder through the foot valve, but substantially preventing drywall joint compound from flowing out of the main cylinder through the bottom end, the foot valve comprising a pin and two semi-circular-shaped rigid flaps that hingedly rotate about the pin.

5. The drywall mud pump of claim 4, the foot valve further comprising a tube, which, when the drywall mud pump is assembled, the pin extends through the tube and the tube extends through a portion of each of the flaps.

6. The drywall mud pump of claim 1 wherein the handle comprises a first member and a second member wherein, when the drywall mud pump is assembled, the first member is pivotably connected to the first end of the rod, and the second member is pivotably connected to the structural component at the pivot point.

7. The drywall mud pump of claim 6 wherein, when the drywall mud pump is assembled, the structural component is rigidly attached to the main cylinder or to the pump head, and the first member slidably and telescopically engages the second member to allow the rod to travel in a substantially straight line along the longitudinal axis while the second member rotates about the pivot point.

8. The drywall mud pump of claim 6 further comprising a bearing mounted within the second member, wherein, when the drywall mud pump is assembled, part of the first member fits inside the bearing and extends into the second member, and wherein the bearing comprises PTFE or multiple balls.

9. A drywall mud pump for pumping drywall joint compound into a drywall tool, the drywall mud pump comprising:
a main cylinder having a top end and an bottom end;
a rod having a longitudinal axis, a first end, and a second end, wherein, when the drywall mud pump is assembled, the second end is located within the main cylinder;
a piston which, when the drywall mud pump is assembled, is located within the main cylinder and is attached to the rod at the second end, the piston comprising at least one orifice therethrough to pass drywall joint compound through the piston when the piston is traveling downward in the main cylinder, and wherein the piston further comprises at least one flapper to block the at least one orifice to substantially prevent passage of drywall joint compound through the piston when the piston is traveling upward in the main cylinder;
a pump head having an output aperture, wherein, when the drywall mud pump is assembled, the pump head is connected to the top end of the main cylinder and the rod passes through the pump head, the pump head comprising a seal around the rod;
a structural component wherein, when the drywall mud pump is assembled, the structural component is attached to the main cylinder or to the pump head and extends to a pivot point; and
a handle wherein, when the drywall mud pump is assembled, the handle is pivotably connected to the first end of the rod, and the handle is pivotably connected to the structural component at the pivot point; and
a foot valve, which, when the drywall mud pump is assembled, is attached to the bottom end of the main cylinder allowing drywall joint compound to flow into the main cylinder through the foot valve, but substantially preventing drywall joint compound from flowing out of the main cylinder through the bottom end, the foot valve comprising a pin and two semi-circular-shaped rigid flaps that hingedly rotate about the pin.

10. The drywall mud pump of claim 9, the foot valve further comprising a tube, wherein, when the drywall mud pump is assembled, the pin extends through the tube and the tube extends through a portion of each of the flaps.

11. The drywall mud pump of claim 9 further comprising a clamp configured to secure the drywall mud pump to a side of a bucket.

12. The drywall mud pump of claim 9 wherein the clamp comprises a force-amplification mechanism and a contact surface to contact an exterior of the bucket, wherein, when the drywall mud pump is assembled and is installed within a bucket, the contact surface faces towards the main cylinder to secure the main cylinder within the bucket by compressing the side of the bucket.

13. The drywall mud pump of claim 9 wherein the clamp comprises a toggle clamp.

14. The drywall mud pump of claim 9 wherein the handle comprises a first member and a second member wherein, when the drywall mud pump is assembled, the first member is pivotably connected to the first end of the rod, and the second member is pivotably connected to the structural component at the pivot point.

15. The drywall mud pump of claim 14 wherein, when the drywall mud pump is assembled, the structural component is rigidly attached to the main cylinder or to the pump head, and the first member slidably and telescopically engages the second member to allow the rod to travel in a substantially straight line along the longitudinal axis while the second member rotates about the pivot point.

16. The drywall mud pump of claim 14 further comprising a bearing mounted within the second member, wherein, when the drywall mud pump is assembled, part of the first member fits inside the bearing and extends into the second member.

17. A method of selecting, obtaining, or providing a drywall mud pump for pumping drywall joint compound from a bucket into a drywall tool, the method comprising in any order at least the acts of:
selecting, obtaining, or providing a body comprising:
an inlet to take in drywall joint compound from the bucket; and
an output aperture to deliver drywall joint compound to the drywall tool;
selecting, obtaining, or providing a driver to move the drywall joint compound through the body;
selecting, obtaining, or providing a structural component wherein, when the drywall mud pump is assembled, the structural component is attached to the body and extends to a pivot point;
selecting, obtaining, or providing a handle wherein, when the drywall mud pump is assembled, the handle is connected in driving relation to the driver, the handle is pivotably connected to the structural component at the pivot point, and the handle rotates about the pivot point; and
selecting, obtaining, or providing a clamp configured to secure the drywall mud pump to a side of a bucket.

18. The method of claim 17 further comprising an act of selecting, obtaining, or providing a force-amplification mechanism and a contact surface to contact an exterior of the bucket, wherein, when the drywall mud pump is assembled, the contact surface faces towards the body to secure the main cylinder within the bucket by compressing the side of the bucket between the contact surface and the body.

19. The method of claim 17 wherein the act of selecting, obtaining, or providing the handle comprises selecting, obtaining, or providing a first member and a second member wherein, when the drywall mud pump is assembled, the first member is pivotably connected to the first end of the rod, the second member is pivotably connected to the structural component at the pivot point, and wherein, when the drywall mud pump is assembled, the structural component is rigidly attached to the body, and the first member slidably and telescopically engages the second member to allow the rod to travel in a substantially straight line along the longitudinal axis while the second member rotates about the pivot point.

20. The method of claim 17 further comprising an act of selecting, obtaining, or providing an inlet valve, which, when the drywall mud pump is assembled, is attached to the inlet to the body allowing drywall joint compound to flow into the inlet and into the body through the inlet valve, but substantially preventing drywall joint compound from flowing out of the body through the inlet, the inlet valve comprising a pin and two semi-circular-shaped rigid flaps that hingedly rotate about the pin.

21. The method of claim 17, wherein the act of selecting, obtaining, or providing the handle further comprises selecting, obtaining, or providing a PTFE bearing.
